(19) United States
(12) Patent Application Publication
(10) Pub. No.: US 2004/0116382 A1
(43) Pub. Date: Jun. 17, 2004

(54) Food products containing cyclodextrins having beneficial hypocholesterolemic effects and method of making and communicating the benefit of such products

(76) Inventors: David W. Plank, Taylors Falls, MN (US); Daniel J. Lewandowski, Bloomington, MN (US)

Correspondence Address: GENERAL MILLS, INC., P.O. BOX 1113, MINNEAPOLIS, MN, 55440, US

(21) Appl. No.: 10/318,445

(22) Filed: Dec. 13, 2002

(51) Int. Cl. A61K 31/724

(52) U.S. Cl. 514/58; 424/439

(57) ABSTRACT

The present invention is related to a novel component for use in a consumer food product that aids in providing beneficial hypocholesterolemic effects. More specifically, the present invention is directed to the use of alpha-cyclodextrin in the preparation of food products to lower harmful cholesterol levels. The food product provides beneficial hypocholesterolemic activity through increased bile acid and lipid binding activity while simultaneously delivering a food product, which is not adversely affected by its inclusion, either in taste or texture or in any undesirable side effects. The invention is also directed to communicating the benefit of the particular food product.

### FIELD OF THE INVENTION

The present invention is related to a novel component for use in a consumer food product that aids in providing beneficial hypocholesterolemic effects. More specifically, the present invention is directed to the use of alpha-cyclodextrin in the preparation of food products to lower harmful cholesterol levels. The food product provides beneficial hypocholesterolemic activity through increased bile acid and lipid binding activity while simultaneously delivering a food product, which is not adversely affected by its inclusion, either in taste or texture or in any undesirable side effects. The invention is also directed to communicating the benefit of the particular food product.

### BACKGROUND OF THE INVENTION

There is a large amount of information in circulation today concerning elevated cholesterol levels and the health consequences due to that condition. In an effort to combat this result, a number of pharmaceutical applications, dietary supplements and other solutions relating to the treatment of high cholesterol levels have been previously introduced. However, regrettably, many of these products can be expensive, have an unpleasant mouth feel, that is they can feel slimy, have a displeasing taste or result in undesirable side effects, which diminishes their overall value to the intended end user.

In addition, there also appears to be a growing disdain against ingesting some sort of dietary supplement, pharmaceutical treatment or other product to attain some perceived beneficial effect from such products. This may be due to a growing reliance on pills or tablets to sustain or maintain our health. Such reliance on supplements may also surprisingly contribute to malnutrition as other valuable vitamins and minerals can be omitted or overlooked when too much focus is diverted to certain items. Moreover, certain supplements may actually remove valuable macronutrients and micronutrients from the system.

Individuals may also be concerned with potential risks and side effects associated with certain medications, treatments or supplements. In fact, dietary restrictions and other health concerns may preclude certain portions of the population from even consuming such products. As such, there remains a continuing interest in developing good tasting, well balanced, food products that contribute to a well balanced diet as well as provide a vehicle by which to deliver the benefit of cholesterol reduction in a palatable and efficient manner to meet the changing needs of the population.

Cholesterol in humans is known to come from primarily two sources, the body's own production of cholesterol (endogenous) and dietary cholesterol (exogenous). Lipoproteins contain specific proteins and varying amounts of cholesterol, triglycerides and phospholipids.

Bile acids are synthesized from cholesterol in the liver and then secreted into the intestines. Reducing the level of bile acid reabsorption facilitates the maintenance of a healthy cholesterol level. One method for reducing bile acid reabsorption is achieved by increasing the gut viscosity. Alternatively, a non-digestible dietary component, which binds bile acids secreted in the proximal jejunum, will reduce bile acid reabsorption in the lower intestines (distal ileum). Additionally, a non-digestible dietary component, which binds lipids (e.g. phoshatidyl choline) may disrupt micelles in the small intestine thereby reducing cholesterol and bile acid uptake. The fermentation of this non-digestible dietary component in the cecum may also play a role in lowering cholesterol levels through the production of short-chain fatty acids and through the acidification of the cecum.

There are three major classes of lipoproteins and they include very low-density lipoproteins (“VLDL”), low-density lipoproteins (“LDL”) and high-density lipoproteins (“HDL”). The LDLs are believed to carry about 60-70% of the serum cholesterol present in an average adult. The HDLs carry around 20-30% of serum cholesterol with the VLDL having around 1-10% of the cholesterol in the serum. To calculate the level of non-HDL cholesterol present (find the level of LDL or VLDL levels), which indicates risk; the HDL is subtracted from the total cholesterol value.

Typically, the average person consumes between 350-400 milligrams of cholesterol daily, while the recommended intake is around 300 milligrams. Increased dietary cholesterol consumption, especially in conjunction with a diet high in saturated fat intake, can result in elevated serum cholesterol. Having an elevated serum cholesterol level is a well-established risk factor for heart disease and therefore there is a need to mitigate the undesired effects of cholesterol accumulation. High cholesterol levels are generally considered to be those total cholesterol levels at 200 milligrams and above or LDL cholesterol levels at 130 milligrams and above. By lowering the total system LDL cholesterol level, it is believed that certain health risks, such as coronary disease and possibly some cancers, that are typically associated with high cholesterol levels, can be reduced by not an insignificant amount.

Numerous studies relating to modifying the intestinal metabolism of lipids have been done to illustrate that such effects can reduce a high cholesterol level. Hampering the absorption of triglycerides, cholesterol or bile acids or a combination of these items results in a lowering of cholesterol levels in the serum.

Soluble dietary fiber is known to be a safe ingredient due to its long history in food supply. Soluble fiber typically remains undigested, except by colonic microflora present in the lower intestines. Soluble dietary fiber is believed to have a beneficial effect in the reduction of high serum cholesterol levels and reducing the risk associated with such elevated levels. In addition, soluble dietary fiber can have the additional beneficial effect of reduced constipation and improved regularity. However, too much fiber in the diet can create undesirable gastrointestinal side effects such as flatulence, diarrhea, and abdominal cramps, etc. leading consumers to stay away from food products that contain too much dietary fiber, regardless of any associated health benefits.

While some consumers may not completely avoid such products, they also do not typically regularly use such products due to the problems enumerated above or alternatively, or in combination due to the unpleasant taste of such products. This illustrates some of the problems with prior solutions that were aimed at providing high fiber diets directed at lowering cholesterol levels, and highlights the need to create a more balanced solution that fits not only within more normal dietary patterns but also meets consumer demand for better tasting, healthy products.

There are a number of other products purporting to have cholesterol-lowering properties available in the market today. One such product offering or solution is described in U.S. Pat. No. 6,136,349 which relates to a food product, food additive or the like that may be fortified with a select group of minerals, such as calcium, magnesium or potassium which when combined with conventional sterols and/or stanols increases the effect of the sterols and/or stanols in lowering cholesterol levels than with just sterols an/or stanols alone. However, significantly increasing only certain nutrients and minerals while ignoring others can result in over consumption or under consumption of essential nutrients because some nutrients are present in very high concentrations while other nutrients are present in very low concentrations. This creates a nutritionally unbalanced situation causing the consumer to either procure the missing macro and/or micronutrients through other food sources or omit them from their diet altogether. In addition to not receiving the DV (Daily Value) of certain nutrients, this may force the consumer into an over compensation mode causing the consumer to ingest more food than is actually necessary thereby defeating the purpose of such cholesterol-lowering foods, and potentially create other problems such as weight gain.

Another possible solution is described in U.S. Pat. No. 6,174,560, which relates to a food composition for lowering low-density cholesterol levels (LDL) and focuses on the use of at least one stanol fatty acid ester in combination with a nutritional substance. The applicants of U.S. Pat. No. 6,174,560 however indicates that increasing the amount of fiber to reduce serum cholesterol levels has been of a limited effect and citing that fiber that is delivered in therapeutically effective doses, such as with pharmaceutical applications, can cause extreme abdominal discomfort. This provides another singular example of a particular element or component being relied upon for a health effect but still ignoring the combined beneficial effects of the present invention as well as the ability to deliver the food product in an acceptable manner.

U.S. Pat. No. 5,244,887 describes the use of stanols as food additives to reduce cholesterol absorption. In the preparation of the additives, sitostanol is dissolved with an edible solubilizing agent such as triglyceride, an antioxidant such as tocopherol, and a dispersant such as lecithin, polysorbate 80, or sodium lauryl sulfate. However, no data is provided in the selection of the most effective components and their amounts or specific methods of preparation. Effectiveness in reducing cholesterol absorption was also not determined. The preferred embodiment consisted of 25% by weight stanols in vegetable oil, but the solubility of sterols in oil is only 2%.

Another difficulty with many of the prior art solutions, regardless of whether they are successful in lowering cholesterol levels or not, is simply a matter of the cost of the ingredients or components which are needed to achieve the desired benefit. Only a very small segment of the population may be willing to pay eight or even ten dollars for a box of cereal or a loaf of bread, despite the benefit associated with it. In addition even if consumers purchase such a product initially, the high cost is likely to be more of a disincentive to purchase the product in the future, when compared with the incentive of the health benefit associated with the product.

Another problem associated with such prior art food problems is that the consumer may be forced to eat several servings of the food product in order to attain the benefit of cholesterol reduction. This further complicates the delivery of the health benefit to the consumer in that a consumer may not want to eat a half a loaf of bread or consume three or more bowls of cereal at a meal. Moreover, over consumption can lead to other problems such as weight gain.

A focus of the present invention relates to a novel use of cyclodextrins, either individually or potentially synergistically, in connection with other ingredients or components to enhance the hypocholesterolemic benefit. Such an unanticipated benefit permits the manufacturer to more efficiently manufacture products, by removing high cost ingredients, which meet the budgetary restrictions of consumers.

Cyclodextrins comprise a doughnut shaped or cyclical structure composed of between six to eight alpha-D-glucose units having a hydrophilic exterior (hydrophilic OH groups on the exterior rim) and a hydrophobic interior (electron dense hydrogen and oxygen atoms). Cyclodextrins are generally water soluble, free flowing crystalline powders that are substantially if not completely odorless and white in color.

Heretofore, starches such as cyclodextrins have not been employed or known for their hypocholesterolemic activity in humans. Cyclodextrins have been used principally for the encapsulation of insoluble compounds on a molecular basis in order to enhance stability, reduce volatility and alter solubility as well as to increase shelf life of certain products. Such prior uses of cyclodextrins have been limited to flavor carriers and protection of sensitive substances against thermal decomposition, oxidation and degradation. In addition, more recently, cyclodextrins have also been used to remove fatty acids and cholesterol from animal fats and to remove cholesterol and cholesterol esters from egg yolks.

Another potential solution to the high cholesterol problem teaches the treatment of the foodstuffs themselves with cyclodextrins rather than the consumer. U.S. Pat. Nos. 5,498,437, 5,342,633 and 5,063,077 discuss various processes for the removal of cholesterol and cholesterol esters from egg yolks, meat, animal fats, etc. It is thought that by reducing the level of cholesterol in such foodstuffs that overall levels of cholesterol may be reduced in consumers. However, processing steps to such foodstuffs increases the cost of delivering such products to market.

Another similar but apparently unrelated reference, which deals with removal of cholesterol from foodstuffs, is U.S. Pat. No. 5,232,725. This reference discusses a process for reducing cholesterol and free fatty acids in an animal fat and the material obtained from that process through the use of cyclodextrins. U.S. Pat. No. 5,223,295 also discusses the use of cyclodextrin to remove steroid based compounds from foodstuffs, particularly egg yolks. However, these patents suffer from the same drawbacks as those referenced above, in that the processing steps required to achieve the result adds another layer to delivering product to the market, causing delay and adding cost.

A further drawback associated with such prior solutions of treating the foodstuff as opposed to treating the consumer, is that the consumer may then be tempted to eat more of the product sensing that the cholesterol level of the product has been reduced, that is the consumer may have a four egg omelet instead of two, thus ingesting the same level of cholesterol as before, thereby defeating the overall purpose of the solution.

PCT Publications WO 99/59421 and WO 99/63841 disclose the use of phytosterols as a pharmaceutical agent or as an addition to certain foodstuffs for lowering cholesterol. The publication discusses that greater effectiveness of the phytosterols can be achieved when using a specified delivery vehicle such as a complexation with cyclodextrins. This represents little more than using cyclodextrins for a purpose that they are already known for, as a carrier for sensitive ingredients.

Another reference that teaches the use of beta-cyclodextrin as a carrier or delivery vehicle is U.S. Pat. No. 4,978,532. In this reference, dehydroepiandrosterone (DHEA) is delivered to the patient via a treatment patch. Beta-cyclodextrin is selected from a group of “permeation enhancers” to facilitate the delivery of the DHEA dose to the patient.

U.S. Pat. No. 5,624,940 the use of various complexes which include cyclodextrins for reducing bone loss and serum cholesterol levels in mammals. In this reference, the cyclodextrin, specifically hydroxypropyl-beta-cyclodextrin is used as a pharmaceutical delivery agent and not as an active ingredient useful in the reduction of serum cholesterol levels.

U.S. Pat. No. 4,877,778 discusses the administration of doses of 2-hydroxypropyl-beta-cyclodextrin at levels of up to 0.5 gm/kg per day. The cyclodextrin is used as a carrier to remove excess lipophiles from the system, specifically as set forth in the example, reduction of high vitamin A levels. With respect to serum cholesterol levels, the '778 patent suggests that the reduction of serum cholesterol levels achieved in the example is due to the system recognizing an overabundance of cholesterol and the serum cholesterol being subsequently “down-regulated. Such down-regulation is a known biologic phenomenon.” The '778 patent goes on to indicate that it is “the natural cholesterol carrying system which predominates and it is the new homeostasis which must be responsible for the observed drop in serum cholesterol.” Hence, the '778 patent does not suggest that the cyclodextrin is usable as a mechanism to bind bile acids or lipids to decrease reabsorption in the lower intestines and is merely cumulative of the prior art which illustrates the use of cyclodextrin as a particular pharmaceutical carrier to treat certain disorders.

Publications, patents and patent applications are referred to throughout this disclosure. All references cited herein are hereby incorporated by reference.

All percentages and ratios are calculated by weight unless otherwise indicated. All percentages and ratios are calculated based on the total composition unless otherwise stated.

### BRIEF SUMMARY OF THE INVENTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

The present invention provides a food product that is palatable and has a good mouth feel and texture so that consumers of the product are not limited solely to those having elevated cholesterol levels, i.e. those total cholesterol levels above 200 mg or those with LDL levels above 130 mg. Consumers with lower cholesterol levels, whether total cholesterol or LDL cholesterol levels, can maintain their average or “good range” cholesterol levels and it is believed, do not experience any adverse effect such as a further lowering of these levels.

In a preferred embodiment of the present invention a food product useful in lowering cholesterol levels, includes a food product having an amount of alpha-cyclodextrin ranging between about 0.01 percent by weight to about 98 percent by weight per serving size and wherein the amount of alpha-cyclodextrin is created through the use of an enzyme while the food product is being processed. The food product suitable for use in lowering cholesterol levels.

In a further embodiment of the present invention a method of communicating a dietary effect of a food product having beneficial hypocholesterolemic activity, is described and includes the steps of initially, providing a food product having an amount of alpha-cyclodextrin, then packaging the food product, next, producing a message relating to the food product and its beneficial hypocholesterolemic activity; and finally distributing the food product to consumers for consumption.

A still further embodiment of the present invention relates to a food product having beneficial hypocholesterolemic activity, that includes an amount of an alpha-cyclodextrin ranging between about 0.01 percent by weight to about percent by weight per serving size; a consumable intermediate composition suitable for use with the amount of alpha-cyclodextrin; and wherein the consumable intermediate composition with the amount of alpha-cyclodextrin provides a food product having beneficial hypocholesterolemic activity.

A yet still further embodiment of the present invention relates to a method of making a food product having a beneficial hypocholesterolemic effect. The method includes the steps of, initially providing a series of pre-selected ingredients and replacing at least a portion of one of the pre-selected ingredient with an amount of alpha-cyclodextrin. Then the pre-selected ingredients are mixed with the amount of alpha-cyclodextrin to create a food intermediate and finally, the food intermediate is further processed to create a food product having a beneficial hypocholesterolemic effect.

### DETAILED DESCRIPTION OF THE INVENTION

The present invention is now illustrated in greater detail by way of the following detailed description, but it should be understood that the present invention is not to be construed as being limited thereto.

As used herein a food product may include an additive, component, supplement or ingredient useful in preparing or supplementing a food, or a food intermediate a fully prepared composition but in a raw state (requiring a further treatment step prior to consumption, such as baking dough to produce bread) or a finished food product that is ready to eat. Food products may also include nutritional beverages and energy drinks.

As used herein a ready to eat food product includes baked goods, muffins, rolls, cakes, pies, crackers, toaster pastries, pastries, grain based bars, granola bars, health food bars, breads, cereals, fruit snacks, fruit bars, pizza rolls, soups, pasta, yogurt, pudding, beverages, sauces, snacks (potato crisps, corn chips, tortilla chips, extruded snacks, enrobed extruded snacks, pretzels), rice and corn cakes.

The term serving size as used herein varies depending on the product, for example with a ready to eat cereal such as CHEERIOS® available from General Mills, Inc. Minneapolis, Minn. 55426, the serving size may range from 30 to 55 grams, a dairy product such as yogurt may have a serving size of ranging from approximately 30 grams up to 225 grams, snack sizes may range from 30 grams to over a 100 grams.

Inclusion of cyclodextrins in ready to eat (RTE) cereals, mixes, doughs, grain based foods and other food products in an amount between 0. 1% to 25% by weight, preferably from 2 to 6 %, can increase the amount of bile acid binding activity occurring in the gut and thereby reduce total serum cholesterol levels. Cyclodextrin is a product of enzymatic conversion or degradation of starch in which a cyclic ring of sugars is created containing between 5 to 1,000,000 glucose units and more typically between 6 to 8 glucose units. A principal source of cyclodextrins is maize starch. However, cyclodextrins may be derived from a wide variety of plant starches. As indicated above, the cyclodextrins have a hydrophobic core that can bind cholesterol or bile acids and allow these molecules to be excreted from the system in the stool.

Enzymatic degradation or treatment of the starch is done by cyclodextrin glucosyltransferase (EC 2.4.1.19) or other enzymes, which results in a cyclic ring of sugar. The cyclodextrin is resistant to digestion but is susceptible to fermentation by bacteria in the cecum or foregut of the organism. The hydrophobic core serves as binding sites for bile acid and steroids, namely cholesterol. The bond formed between the cyclodextrin and the bile acids and cholesterol is sufficiently strong so as to enable the material to pass through the system without being reabsorbed through the intestines.

The preferred starch of the present invention are cyclodextrins, preferably alpha-cyclodextrins. As indicated previously, cyclodextrins comprise a doughnut shaped or cyclical structure composed of a number of alpha-D-glucose units (typically 6-8) having a hydrophilic exterior and a hydrophobic interior. Alpha-cyclodextrin is a cyclized ring of six alpha 1,4 linked glucose units.

Cyclodextrins are generally water soluble, although alpha-cyclodextrin is likely more water soluble than beta-cyclodextrin or gamma-cyclodextrin, and free flowing crystalline powers that are substantially if not completely odorless and white in color. Heretofore, modified starches such as cyclodextrin were not employed or known for their hypocholesterolemic activity and have been used principally for the encapsulation of insoluble compounds to enhance stability, reduce volatility and alter solubility. Such prior uses of cyclodextrins have been limited to carriers for flavors, therapeutic agents and to remove fatty acids and cholesterol from animal fats.

Alpha-cyclodextrins has a cavity dimension of about 0.50×0.79 (nm) and a cavity volume of about 0.10 (ml). The solubility of alpha-cyclodextrin at 25° C. is 14 (gm/100 mil). Alpha-cyclodextrin is available from Wacker Specialties, Adrian, Mich. 49221 and sold under the trademark CAVAMAX® Wacker-Chemie, Burghausen, Germany.

Other cyclodextrins may be used in combination or synergistically with alpha-cyclodextrin, such as beta-cyclodextrin and gamma-cyclodextrin, in particular ratios dependent upon the requirements of the manufacturer. In an exemplary embodiment, alpha-cyclodextrin is combined with between 0-2% by weight beta-cyclodextrin.

The processing of ready to eat cereals or other food products is done by incubation of the food product with the enzyme, cyclodextrin glucosyltransferase (GCTase) (EC 2.4.19). GCTase is a member of the alpha-amylase family of enzymes. The enzyme is provided at between the levels of 0.00001 to 50.0% weight of the product. By incubating the food product or components of the food product during the processing with the enzyme, polysaccharides are converted into non-digestible, fermentable forms that increase the efficacy of the food product to lower the ingesting organisms cholesterol concentration.

### EXAMPLE 1

In an exemplary embodiment of the present invention, the food product, in this case a ready to eat (RTE) cereal, may include the following micro and macronutrients in connection with an amount of alpha-cyclodextrin. In this example, about 1-3 gms of alpha-cyclodexrin is used. The serving size ranges from 30 to 55 grams.

Recommended Daily Value (“DV”)

| | |
|---|---|
| Total Fat | 5% |
| Saturated Fat | 3% |
| Cholesterol | 0% |
| Sodium | 10% |
| Potassium | 5% |
| Total Carbohydrate | 14% |
| Dietary Fiber | 17% |
| Protein | 10% |
| Vitamin A | 10% |
| Vitamin C | 70% |
| Calcium | 0% |
| Iron | 80% |
| Vitamin D | 10% |
| Vitamin E | 100% |
| Thiamin | 100% |
| Riboflavin | 80% |
| Niacin | 80% |
| Vitamin B6 | 80% |
| Folic Acid | 100% |
| Vitamin B12 | 100% |
| Phosphorus | 15% |
| Magnesium | 10% |
| Zinc | 80% |
| Copper | 4% |

This example is based on a 2,000 calorie diet and other food products such as cereal bars, fruit snacks, diary and bakery products, baking mixes and ready to eat meals may contain additional vitamins, nutrients and or minerals as well as potentially varying amounts of the macro and micro nutrients set forth in the instant example.

### EXAMPLE 2

In the second example the nutritionally complete food product, in this case a ready to eat (RTE) cereal, may include the following micro and macronutrients in connection with a high alpha-cyclodextrin based formula. In this example, about 2-10 grams of alpha-cyclodextrin is used. The serving size is approximately 55 grams.

Recommended Daily Value (“DV”)

| | |
|---|---|
| Total Fat | 4% |
| Saturated Fat | 3% |
| Cholesterol | 0% |
| Sodium | 10% |
| Potassium | 5% |
| Total Carbohydrate | 26% |
| Dietary Fiber | 5% |
| Protein | 10% |
| Vitamin A | 10% |
| Vitamin C | 70% |
| Calcium | 0% |
| Iron | 80% |
| Vitamin D | 10% |
| Vitamin E | 100% |
| Thiamin | 100% |
| Riboflavin | 80% |
| Niacin | 80% |
| Vitamin B6 | 80% |
| Folic Acid | 100% |
| Vitamin B12 | 100% |
| Phosphorus | 15% |
| Magnesium | 10% |
| Zinc | 80% |
| Copper | 4% |

This example is based on a 2,000 calorie diet and other food products such as cereal bars, fruit snacks, diary and bakery products baking mixes and ready to eat meals may contain additional vitamins, nutrients and or minerals as well as potentially varying amounts of the macro and micro nutrients set forth in the instant example.

The RTE cereals of the preceding examples are prepared in a conventional manner, mixing the ingredients, except that a portion of the flour (oat, wheat) is replaced with alpha-cyclodextrin or the ingredients are first processed with an enzyme that produces alpha-cyclodextrin such as cyclodextrin glucosyltransferase to produce a food intermediate, here a dough. This exemplary RTE cereal is in the form of flakes that are created by preparing a cooked cereal dough through known methods and then forming the cooked cereal dough into pellets that have a desired moisture content. The pellets are then formed into wet flakes by passing the pellets through chilled roller and then subsequently toasting or heating the wet cereal flakes. The toasting causes a final drying of the wet flakes, resulting in slightly expanded and crisp RTE cereal flakes. The flakes are then screened for size uniformity. The final flake cereal attributes of appearance, flavor, texture, inter alia, are all affected by the selection and practice of the steps employed in their methods of preparation. For example, to provide flake cereals having a desired appearance feature of grain bits appearing on the flakes, one approach is to topically apply the grain bits onto the surface of the flake as part of a coating that is applied after toasting.

While the foregoing examples are directed to the manufacture of flake cereals, it is readily apparent, that the manufacturing method can be modified to produce puffed or extruded cereals as well in which the dough after forming is either fed through an extruder to create the desired shape or in the alternative is forced through a “gun” to generate puffed cereals.

In addition to the beneficial health aspects of including alpha-cyclodextrin in the ready to eat food product produced in accordance with the above examples, test subjects when sampling CHEERIOS® noticed an improvement in the toasted oat flavor. It is believed that the alpha-cyclodextrin enhanced or accentuated the toasted oat flavor of the food product as opposed to carrying flavors that not may not necessarily be inherent in the product as may have been done with other cyclodextrins in the prior art.

Cereal products prepared in connection with the above-mentioned example 1 were then fed to hamsters and the results compared. A control feed, a feed which is principally an oat cereal, an oat cereal in which a portion of the oat flour has been replace with 3.5% concentration of alpha-cyclodextrin. The results of this study and provided for approximately a 15% reduction in the total cholesterol level as well as the HDL level between the oat cereal and the oat cereal with alpha-cyclodextrin.

The results of the study also provides that the food product of the present invention resulted in a lowering of triglycerides of about ten percent (10%) when compared with the level of triglycerides in the control formula.

In a further example of the present invention a consumable intermediate (dough which is used for making bakery products) was prepared. As provided previously, the intermediate requires further treatment in order to be ready for consumption. Such additional treatment may include but is not limited to cooking, heating, baking, frying, applying radiant energy, etc.

The dough mixture used in forming the dough intermediate was prepared in accordance with the following. The formula is intended to be illustrative only and not limiting in scope of the present invention.

### EXAMPLE 3

| Ingredient | Weight Percentage |
|---|---|
| Flour | 51.8 |
| Water | 23.98 |
| Sugar | 4.03 |
| Corn Syrup | 3.73 |
| Dextrose | 3.51 |
| Yeast | 2.09 |
| Glycerol | 1.86 |
| Shortening | 1.84 |
| Egg Solids | 1.77 |
| Whey | 1.49 |
| Soda | 0.80 |
| Salt | 0.75 |
| SAPP | 0.55 |
| Mono&DI Glycerides | 0.50 |
| Dough Conditioners | 0.50 |
| Flavor | 0.41 |
| SALP | 0.40 |
| Total | 100 |

As used herein SAPP refers to sodium aluminum pyrophosphates, which is a fast acting chemical leavening agent. SALP refers to sodium aluminum phosphate which is slow acting chemical agent. However, other chemical leavening agents may also be used such as DCP—dicalcium phosphate, MCP—monocalcium phosphate monohydrate, SAS—sodium aluminum sulfate, potassium hydrogen tartrate—cream of tartar, combinations and the like.

The flour is preferably a wheat-based flour, but other flour types such as barley, rice, corn, potato and soy flour may also be used in this invention. As in the above examples, a portion of the wheat-based flour is replaced with alpha-cyclodextrin or the ingredients are first processed with an enzyme that produces alpha-cyclodextrin such as cyclodextrin glucosyltransferase, to produce a food intermediate, here a dough, to arrive at the between 0.01 to 25% by weight of the anticipated serving size of the product.

Other components for dough that are useable in the present invention include, for example fat or shortening in an amount from 1 to 20% by weight, egg solids in an amount of from about 0.01% to about 25%, milk replacer, milk solids or whey in an amount of from about 0.1% to about 12%, sugar in an amount from about 1% to about 25%, yeast in an amount of from about 1.0% to about 7% and water in an amount from about 40% to about 80%. The forgoing percentages are based on weight of the mixture.

The dough was prepared by adding the ingredients to a mixer, where it was mixed on low speed for approximately one minute, until a dough ball was formed and then on medium to high speed for approximately eight minutes.

The dough was then removed from the mixture and then sheeted or rolled out and cut into strips. The ends of the strips were moistened with water to form a sealing end for the product. A filling layer, such as cinnamon, fruit filling, cheese, etc. may be deposited onto the dough strips and either encased in the dough or the dough layers simply wrapped on themselves. The dough was then rolled onto itself to form the desired number of rolls, layers or swirls and then the roll is cut into roughly one inch slices to form the dough intermediate.

Often and potentially more important than the actual manufacture or delivery of a product is communicating the benefits associated with a particular food product to the consumers. This can be done in a number of ways such as through the preparation of scripted information or indicia that is then released to consumers. The release of such indicia is usually tailored to certain pre-selected or predefined formats and can be done through traditional advertising routes that have at least an audio and or visual capability such as radio and television as well printed materials. Printed materials may include the packaging into which the product is placed as well as newspapers, letters, direct mail pieces, magazines and the like.

It will thus be seen according to the present invention a highly advantageous food product has been provided. While the invention has been described in connection with what is presently considered to be the most practical and preferred embodiment, it will be apparent to those of ordinary skill in the art that the invention is not to be limited to the disclosed embodiment, that many modifications and equivalent arrangements may be made thereof within the scope of the invention, which scope is to be accorded the broadest interpretation of the appended claims so as to encompass all equivalent structures and products.

1. A food product useful in lowering cholesterol levels, comprising;
a food product having an amount of alpha-cyclodextrin ranging between about 0.01 percent by weight to about 25 percent by weight per serving size; and
wherein said amount of alpha-cyclodextrin is created using an enzyme while the food product is being processed and said food product suitable for use in lowering cholesterol levels.

2. A food product as recited in claim 1, wherein said enzyme is cyclodextrin glucosyltransferase.

3. A food product as recited in claim 1, wherein said food product also includes an amount of beta-cyclodextrin.

4. A food product as recited in claim 3, where said amount of beta-cyclodextrin is provided in an amount ranging from 0 to 2% by weight.

5. A food product as recited in claim 1, wherein said food product also includes an amount of gamma-cyclodextrin.

6. A food product as recited in claim 1, wherein said food product also includes at least one micronutrient.

7. A food product as recited in claim 1, wherein said food product also includes at least one additional macronutrient.

8. A food product as recited in claim 1, wherein said food is a ready to eat food product.

9. A food product having beneficial hypocholesterolemic activity, comprising;
an amount of an alpha-cyclodextrin ranging between about 0.01 percent by weight to about 25 percent by weight per serving size;
a consumable intermediate composition suitable for use with said amount of alpha-cyclodextrin; and
wherein said consumable intermediate composition with said amount of alpha-cyclodextrin provides a food product having beneficial hypocholesterolemic activity.

10. A food product having beneficial hypocholesterolemic activity as recited in claim 9, wherein said food product is a ready to eat food product.

11. A food product having beneficial hypocholesterolemic activity as recited in claim 9, wherein said consumable intermediate is a dough.

12. A method of communicating a dietary effect of a food product having beneficial hypocholesterolemic activity, comprising the steps of;
providing a food product having an amount of alpha-cyclodextrin;
packaging said food product;
producing a message relating to said food product and its beneficial hypocholesterolemic activity; and
distributing said food product to consumers for consumption.

13. A method of communicating a dietary effect as recited in claim 11, including a further step of printing the message on the packaging for said food product after the step of producing the message.

14. The method of communicating a dietary effect as recited in claim 11, including a further step of creating advertising for the message after the step of producing the message.

15. A food product having a beneficial hypocholesterolemic effect containing between 0.01 and 25% by weight per serving size of alpha-cyclodextrin.

16. A method for producing a food product having a beneficial hypocholesterolemic effect, comprising the steps of;
providing a series of pre-selected ingredients;
replacing at least a portion of one of said pre-selected ingredient with an amount of alpha-cyclodextrin;
mixing said pre-selected ingredients and said amount of alpha-cyclodextrin to create a food intermediate; and
processing said food intermediate to create a food product having a beneficial hypocholesterolemic effect.

17. A method for producing a food product as recited in claim 16, wherein said amount of alpha-cyclodextrin ranges from between about 0.01 to 25 percent of a serving size of the food product.

18. A method for producing a food product as recited in claim 16, wherein said amount of alpha-cyclodextrin is produced through use of an enzyme.

19. A method for producing a food product as recited in claim 16, including a further step of controlling the amount of alpha-cyclodextrin by processing the pre-selected ingredients with an enzyme after the step of providing pre-selected ingredients.

20. A method for producing a food product as recited in claim 19, wherein the enzyme is cyclodextrin glucosyltransferase.
